(19) United States
(12) Patent Application Publication
(10) Pub. No.: US 2024/0148984 A1
(43) Pub. Date: May 9, 2024

(54) Needle Assembly With Integrated Curvature Mechanism

(71) Applicant: United States Government As Represented By The Department Of Veterans Affairs, Washington, DC (US)

(72) Inventor: Ryan McCarty, Los Angeles, CA (US)

(21) Appl. No.: 18/502,774

(22) Filed: Nov. 6, 2023

Related U.S. Application Data
(60) Provisional application No. 63/423,030, filed on Nov. 6, 2022.

(51) Int. Cl. A61M 5/32

(52) U.S. Cl. CPC A61M 5/3286

(57) ABSTRACT

A sheath assembly for use with an elongated needle may include an elongated body portion configured to slidably receive the elongated needle therein, wherein the elongated sheath includes a top surface, a bottom surface, a proximal end, a distal end, and at least one joint portion formed adjacent or proximate the distal end of the sheath. Preferably, at least one joint portion is disposed adjacent or proximate the distal end of the sheath so that a bend in the elongated sheath at least one joint portion produces a corresponding bend in the needle. As such, the needle remains in the sterile environment of the sheath during the bending process, the sheath protecting the physician from needle pricks.

### CROSS-REFERENCE TO RELATED APPLICATION

This application claims priority to and the benefit of the filing date of U.S. Provisional Patent Application No. 63/423,030, which was filed on Nov. 6, 2022, the entirety of which is incorporated herein by reference.

### FIELD

The present application is generally related to needle assemblies and, more particularly, to sheath assemblies for use with elongated needles and methods of use thereof for allowing an individual to impart a desired amount of bend to the elongated needle.

### BACKGROUND

Needles used in medical procedures are generally straight with no curvature at the tip. As an example, the Quincke needle, commonly used in transforaminal epidural steroid injections, is straight with no curvature at the tip. Quincke needles, and many other needles used in medical procedures, have a bevel formed at the tip to facilitate passage through tissues with reduced force required to be exerted on the needle. Within human tissue, the Quincke needle naturally moves in the direction of the side of the needle on which the pointed apex of the bevel is formed, rather than toward the side on which the heel of the bevel is formed. As such, the direction of travel is away from the bevel.

When performing procedures, an interventionist may encounter a bony obstruction and may need to maneuver around the obstruction. To enhance bevel control and improve needle steerability to avoid obstructions, the user may bend the tip of the needle at a 5 to 10 degree angle, or other degree angle as desired. The tip of the needle is bent toward the bevel to enable the user to have improved directional control. A more pronounced bend is used when greater maneuverability is required. Compared to a needle with no bend at the tip, the bent needle tip may not have to be retracted and redirected, thereby reducing tissue damage, pain, and time to complete a procedure. An unbent needle tip is harder to redirect once at depth within the patient as well. The preference for the directional bend of a needle may vary for other procedures or uses. Other standard needles may also be bent at the tip for certain procedures or preferences to increase maneuverability and avoid obstructions. For standard needles that do not have a bevel at the tip, there may be no directional preference for the needle bend.

Typically, a bend formed at the tip of the needle is most effective in the range of 0.5 to 1.0 cm from the tip to the needle. Therefore, it is important that the curvature or bend of the needle be placed in close proximity to the tip of the needle to enhance maneuverability with the remainder of the needle remaining intact and unbent. The desired distance of the bend from the tip of the needle may vary. Needles used in medical procedures are often packaged in an outer plastic sheath to protect the integrity of the needle and handler from accidental needle pricks. Bending the needle tip has historically been performed manually after the needle has been removed from the corresponding sheath. Manually bending the needle may result in the needle tip being bent too little or too much. Manually bending the needle tip may also result in a less standardized and less uniform needle bending process and create a risk that the interventionalist will inadvertently perforate his or her glove and/or own skin while performing the bend.

Additional issues with manually bending a needle tip may include an inexact and non-reproducible nature of the bend as there is no guide as to how much the needle has been bent, the potential “off-axis” nature of the bend as bending may unintentionally take place in two axes given the way the user grasps the needle tip, risk of damage to the needle itself (especially the critical cutting surfaces of the needle bevel or tip), the cost of additional surgical tools, cleaning, and sterilization cycles for each and every procedure, and lost time for the physician and support staff. Furthermore, the forces required to bend a needle containing a stylet may exceed those achievable by using finger pressure alone. Although a practitioner may purchase specially pre-bent needles, these needles are often more expensive and they do not give the option for a straight needle tip in the same packaging.

From the foregoing, one recognizes the need for assemblies and methods of use thereof that may be utilized by individuals to safely and effectively put a desired amount of bend in an elongated needle in a sterile fashion. Having a more standardized process for bending the needle tip, and a more precise way to bend the needle tip, may allow the practitioner to more efficiently maneuver around objects and, for some procedures, use less fluoroscopic radiation and reduce procedure time.

### SUMMARY

Needles used in medical procedures are packaged in an outer sheath to protect both the integrity of the needle and the handler from accidental needle pricks. The sheath typically comprises a hollow (optionally, cylindrical) apparatus into which the needle is placed, and most often is formed from plastic. As noted, for certain procedures, practitioners may bend the tip of the needle prior to the injection to increase the steerability of the needle within the patient's tissue after perforation of the tissue. The present disclosure relates to the bending of the tip of a needle for use in such procedures by way of incorporating a curvature mechanism such as a bendable hinge, joint, living hinge, etc., into the sheath. The practitioner can bend the needle at a customized angle prior to the needle being removed from the sheath. A bent needle tip may be easier to redirect once at depth allowing the practitioner increased maneuverability during the procedure. Additionally, the bendable hinge, joint, etc., allows for repeatability and, therefore, a predictable amount of bend that is imported to the needle, as opposed to when no guidance is provided.

Embodiments of the present disclosure comprise a curvature mechanism incorporated into the needle sheath to allow the practitioner to bend the needle at a customized angle prior to removing the needle from the sheath, the bend being a pre-selected distance (e.g., at a predetermined location) from the tip of the needle. Preferably, the distal end of the needle sheath (in which the tip of the needle is disposed) is tapered or compressed to allow the practitioner to more precisely grasp and bend the needle tip. Various embodiments may include multiple bend lines, grooves, or hinges to allow the practitioner to bend the needle at any of the possible locations.

Embodiments of the present disclosure may also provide a needle assembly that facilitates safely and accurately providing a custom bend in a needle (e.g., an elongated needle) in a sterile manner. Some embodiments include an elongated needle and an elongated sheath configured to slidably receive the elongated needle therein. The elongated sheath may include a top surface, a bottom surface, a proximal end, a distal end, and at least one joint portion formed adjacent or proximate the distal end of the sheath. The at least one joint portion is disposed adjacent or proximate the distal end of the sheath so that a bend in the elongated sheath at the at least one joint portion produces a bend in the needle while the needle remains in the sterile environment of the sheath.

Yet other embodiments of the present disclosure provide a sheath assembly for use with an elongated needle. The sheath assembly may include an elongated sheath configured to slidably receive the elongated needle therein, wherein the elongated sheath includes a top surface, a bottom surface, a proximal end, a distal end, and at least the joint portion formed adjacent or proximate the distal end of the sheath. Preferably, at least one joint portion is disposed adjacent or proximate the distal end of the sheath so that a bend in the elongated sheath at the least one joint portion produces a corresponding bend in the needle. As such, the needle remains in the sterile environment of the sheath during the bending process, with the sheath protecting the physician from needle pricks.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended:

FIGS. 1A, 1B, and 1C are perspective, top, and side views of a needle assembly including a sheath with an integrated curvature mechanism in accordance with an embodiment of the present disclosure;

FIG. 2 is a perspective view the needle assembly including a sheath with an integrated curvature mechanism shown in FIGS. 1A, 1B, and 1C, with the needle sheath bent at the integrated curvature mechanism;

FIGS. 3A and 3B are partial side views of the needle assembly shown in FIGS. 1A, 1B, and 1C with the sheath in the unbent and bent positions, respectively;

FIG. 4 is a perspective view of a needle assembly including a sheath with a pair of integrated curvature mechanisms in accordance with an alternate embodiment of the present disclosure;

FIGS. 5A and 5B are partial side views of the needle assembly including a sheath shown in FIG. 4 with the sheath in the unbent and bent positions, respectively;

FIGS. 6A, 6B, and 6C are perspective views of a needle assembly including a sheath with an integrated curvature mechanism in accordance with an alternate embodiment of the present disclosure; and

FIG. 7 is a partial cross-sectional view of the needle assembly in FIGS. 6A, 6B, and 6C.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” can optionally include plural referents unless the context clearly dictates otherwise. For example, use of the term “a bend” can represent disclosure of embodiments in which only a single such bend is provided, and in alternative aspects, can represent disclosure of embodiments in which a plurality of such bends are provided.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, can in additional alternative aspects, also include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

As further disclosed herein, it is contemplated that incorporating a bendable design element into the sheath of a needle assembly can allow a clinician or other practitioner to bend a needle at a customized angle prior to the needle being removed from the sheath. It is further contemplated that bending a needle while the needle remains in the sheath may increase standardization of the bend, potentially reduce needle sticks, and maintain the sterile environment of the needle.

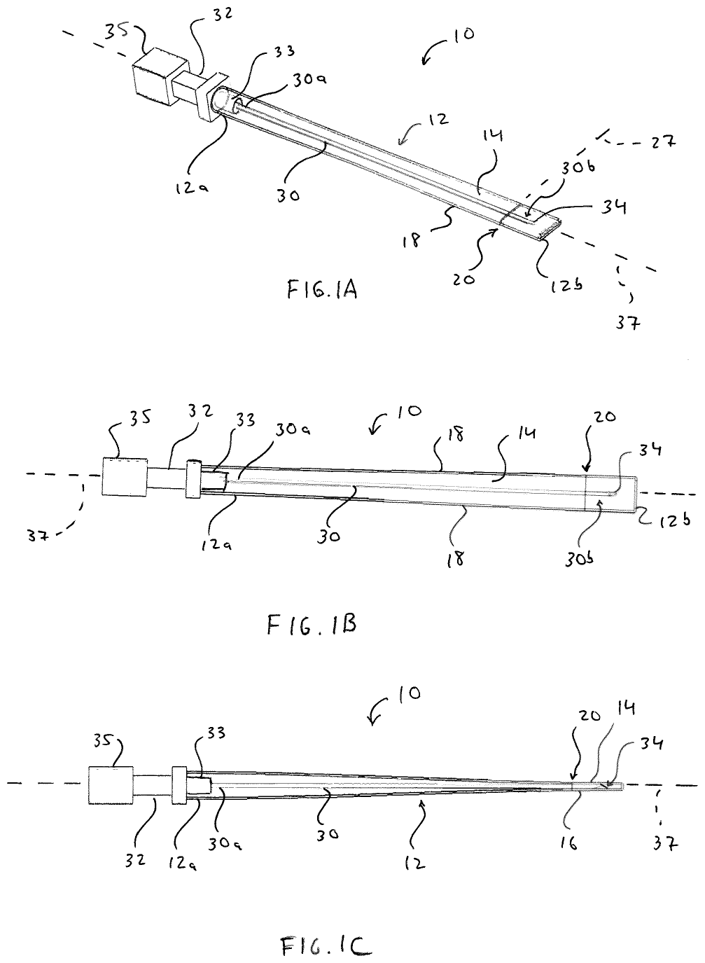

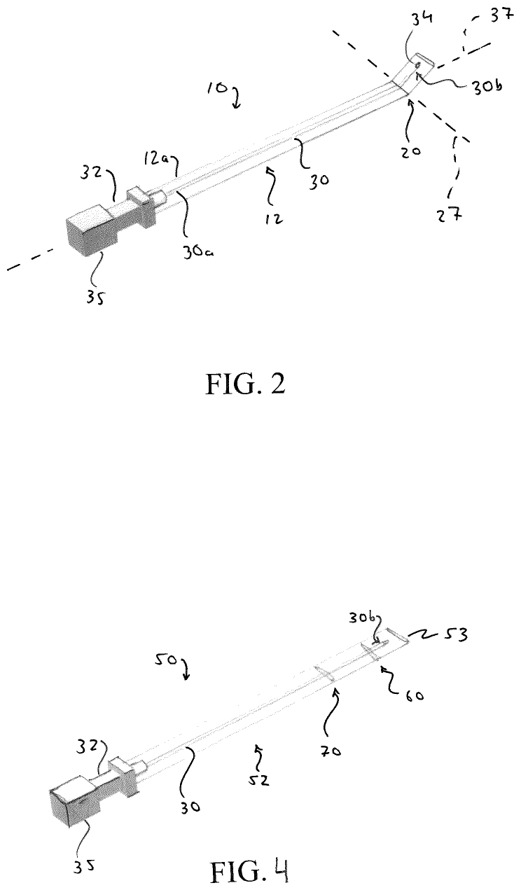

The present disclosure relates generally to sheath assemblies, as shown in FIGS. 1A, 1B and 1C, and methods of use thereof for bending needles. More specifically, the present disclosure relates to an assembly and method for bending needles (e.g., spinal needles that are specially constructed needles that may contain an indwelling metal stylet). These needles can be inserted into the spine as well as other body tissues at a predetermined angle to facilitate steering the tip to a desired target, typically aided by intermittent fluoroscopic guidance. The indwelling metal stylet or wire fills the internal diameter of the spinal needle during insertion in the body, preventing tissue or fluid blockage. Once the tip of the needle is at its intended position, the stylet is removed and an anesthetic, steroid or contrast agent may be injected, or spinal fluid removed, as is done for lumbar puncture.

Certain therapeutic and diagnostic medical procedures involve the use of long spinal needles that may be pre-bent near the tip to facilitate steering the tip of the needle towards an intended target. Other needles may not be pre-bent although a bevel at the tip would prove useful for the practitioner. For those needles, embodiments of the present disclosure can allow a practitioner to impart a predictable, repeatable amount of bend on the tip of the needle.

Referring now to FIGS. 1A, 1B, and 1C, the needle assembly 10 including a needle sheath assembly 12 having an integrated curvature mechanism in accordance with an embodiment of the present disclosure is shown. The needle assembly 10 includes an elongated hollow needle 30 including a proximal end 30a that is secured to a needle hub 32, and the distal end 30b, or tip, that includes a bevel 34. A stylet (not shown) is threaded into the hollow interior of the needle 30 with the back end of the stylet, typically a thin metal wire, being contained in a stylet hub 35 that enables the user to withdraw the stylet once the distal end 30b of the needle 30 has been positioned as desired. Similarly, the needle hub 32 enables the user to grip the needle hub 32 and direct the movement of the distal end 30b of the needle 30 to its intended position. In exemplary aspects, an injection device (e.g., a vial containing a composition to be delivered to a patient) can be positioned in fluid communication with the proximal end of the elongated needle using conventional methods.

A sheath 12 is provided to maintain a sterile environment for the needle 30 and prevent unintended damage to the needle and/or injuries caused by the sharpened distal end 30b. In the embodiment shown, the sheath 12 comprises a substantially hollow body portion that is configured to slidably receive the needle 30 therein. Preferably, the sheath 12 includes a proximal end 12a that is shaped correspondingly to a base portion 33 of the needle hub 32 so that it may be slidably received thereon, and a distal end 12b that is configured to enclose the distal end 30b of the needle 30 therein. As best seen in FIG. 1C, whereas the proximal end 12a of the body portion of the sheath 12 is cylindrical in shape, in keeping with the cylindrical shape of the base portion 33 of the needle hub 32, the body portion of the sheath 12 transitions into a substantially flat distal portion 12a. The distal end 12a of the sheath 12 includes the curvature mechanism 20 in the form of a hinge, slit, groove, combination thereof, or the like to facilitate imparting a predictable amount of bend in the distal end 30b of the needle 30. In the present embodiment, the curvature mechanism of the sheath assembly 12 is preferably formed by a V-shaped groove 20. Optionally, it is contemplated that the sheath can comprise transparent or translucent material to permit visibility of the needle. In exemplary aspects, it is contemplated that the curvature mechanism can be spaced from the distal tip of the sheath 12 by about 0.5 cm to about 3 cm.

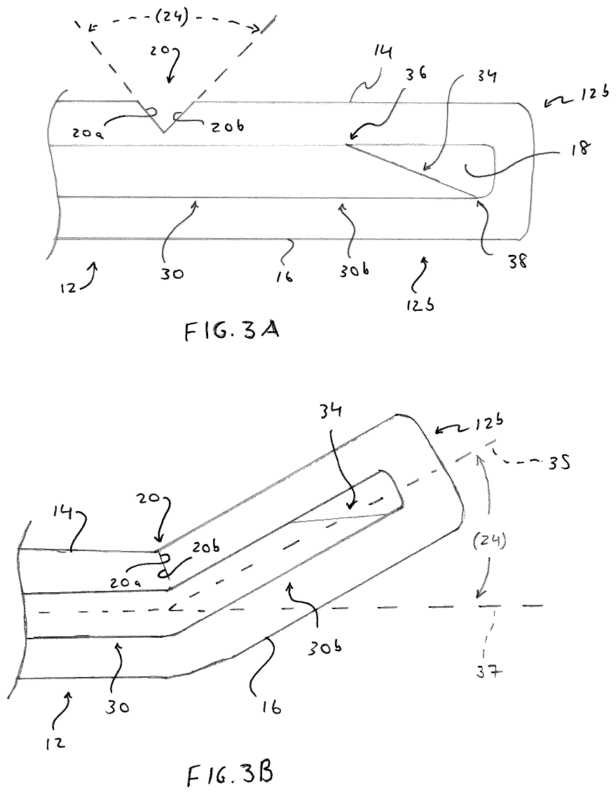

Referring additionally to FIG. 3A, the distal end 12b of the sheath 12 optionally includes a top wall 14 and a bottom wall 16 that are substantially parallel to each other, and a pair of side walls 18 that extend between the side edges of the top and bottom walls 14 and 16. As such, the distal end 12b of the sheath 12 is substantially rectangular in shape. Preferably, the height of the interior cavity of the distal end 12b of the sheath 12 is substantially the same as the outer diameter of the distal end 30b of the needle 30. As such, the distal end 30b of the needle 30 is held in such a manner that there is little to no movement of the distal end 30b of the needle 30 in a vertical direction between the top and bottom walls 14 and 16 of the distal end 12b of the sheath 12. As shown in FIG. 3A, the groove (e.g., V-shaped groove 20) that forms the curvature mechanism of the sheath 12 depends inwardly from an outer surface of the top wall 14 of the sheath 12 in the vicinity of the distal end 12b of the sheath 12. The V-shaped groove 20 can be formed by a pair of intersecting side walls 22a and 22b that define an interior angle 24 therebetween that is preferably a pre-selected angle of less than or equal to 10 degrees (e.g., from about 2 degrees to about 10 degrees). Note, in alternative embodiments, it is contemplated that the interior angle may be selected over numerous ranges dependent upon the amount of a resultant bend that is desired in the distal end 30b of the needle 30. For example, an interior angle of 15 degrees may be selected wherein the resultant bend in the needle 30 is desired to be 15 degrees.

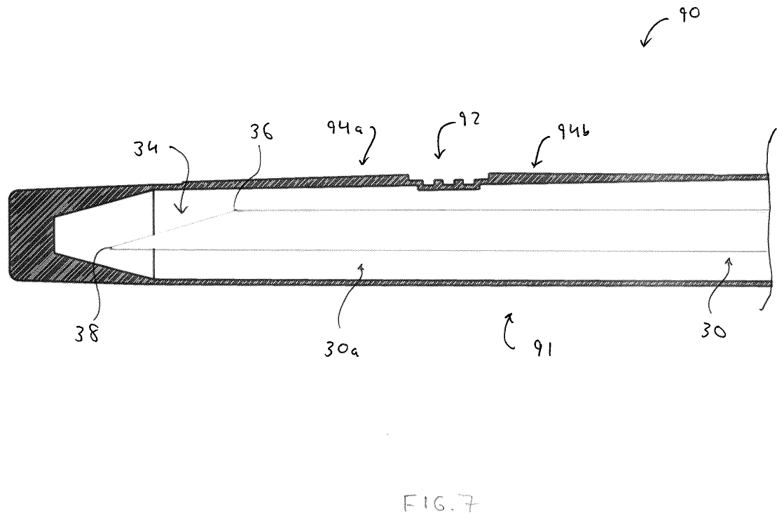

Referring additionally to FIGS. 3B and 6, the distal end 30b of the needle 30 can include a bevel 34 having a heel 36 and an apex 38 that is disposed toward the top wall 14 of the distal end 12b of the sheath 12. This positioning of the bevel 34 is desired due to the fact that the bend imparted on the needle 30 is to be on the side of the needle 30 that includes the heel 36 of the bevel 34. When the user wants to impart the bend on the distal end 30b of the needle 30, the user may position the thumbs or forefingers of the user's left and right hands on the top wall 14 of the sheath 12 on opposite sides of the groove 20. The user next bends the two portions of the sheath 12 inwardly until the side walls 22a and 22b that define the V-shaped groove 20 are in abutment. At this point, the two portions of the sheath 12 that are disposed on opposite sides of the V-shaped groove 20 are now bent so that the longitudinal axis is 35 of the tip of the needle forms an angle with the longitudinal axis 37 of the proximal portion 30a of the needle 30 that equals the interior angle 24 defined between the side walls 22a and 22b of the V-shaped groove when the sheath 12 was in the un-bent position. The user may now relieve force on the sheath 12 and the metal needle 30 will remain in the bent position with the distal end 30b forming the desired angle with respect to the longitudinal axis 37 of the proximal end 30a. The user may now remove the sheath 12 and begin the desired procedure.

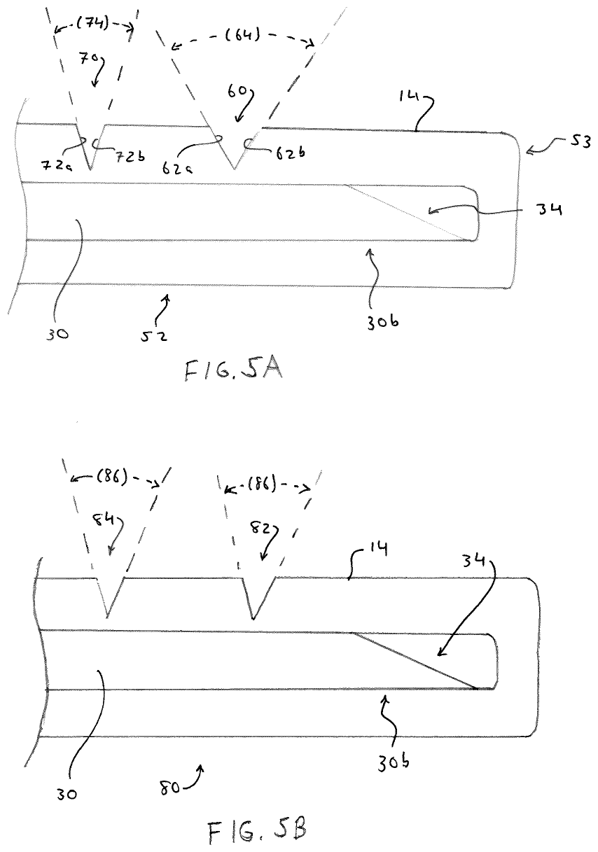

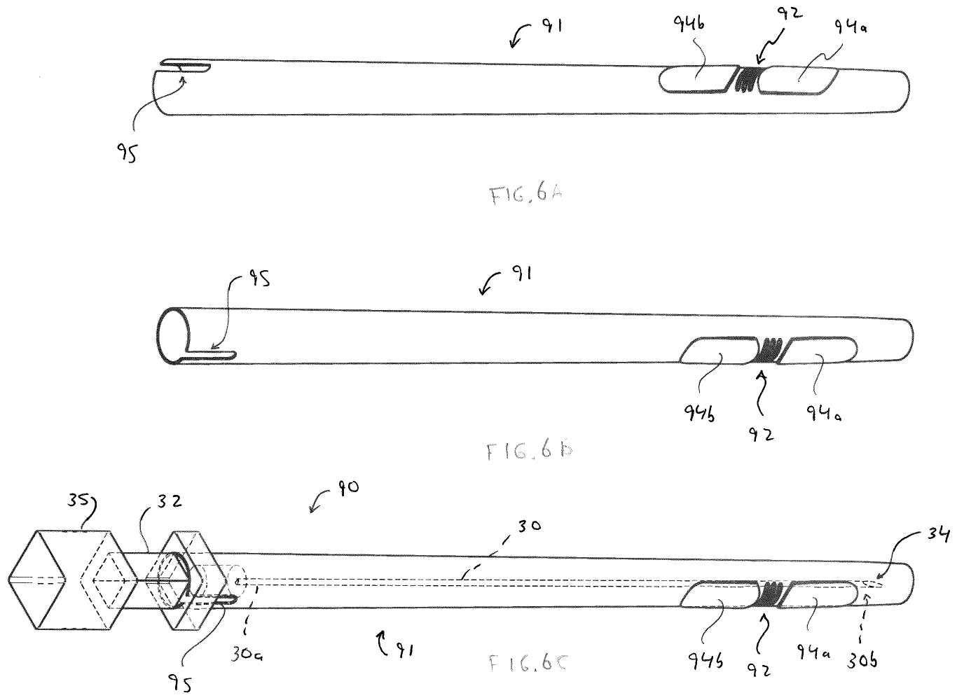

Referring now to FIGS. 4, 5A, and 5B, in an alternative embodiment of a needle assembly 50 including a sheath 52 in accordance with the present disclosure, the sheath 52 may include a first curvature mechanism 60 disposed a first distance from the distal end 53 of the sheath 52 (e.g., about 0.5 cm to about 1.5 cm from the distal end) and a second curvature mechanism 70 disposed a second (greater) distance from the distal end 53 of the sheath 52 (e.g., about 1 cm to about 3 cm from the distal end). As such, a practitioner may form a bend in the distal end 30b of the needle 30 at either of the potential locations. In the present embodiment, the first curvature mechanism is formed by a first groove (e.g., a first V-shaped groove 60) that is defined by a pair of intersecting side walls 62a and 62b that define an interior angle 64 therebetween, whereas the second curvature mechanism is formed by a second groove (e.g., a second V-shaped groove 70) and is defined by a pair of intersecting side walls 72a and 72b that define an interior angle therebetween 74. In the present example, shown in FIGS. 4 and 5A, the first interior angle 64 may be on the order of ten degrees (10°) or range from about 8 degrees to about 12 degrees, whereas the second interior angle 74 may be on the order of five degrees (5°) or range from about 2 degrees to about 8 degrees. Dependent upon the distance of the first V-shaped groove 60 and the second V-shaped groove 70 from the distal end 53 of the sheath 52, the lesser second interior angle 74 may result in the same radial displacement of the tip 30b of the needle 30 from the longitudinal axis 37 as would the larger interior angle 64 that is located in closer proximity to the distal end 53 of the sheath 52. In short, for a given interior angle defined by the side walls of a curvature mechanism, as the curvature mechanism is moved rearwardly away from the distal end of the sheath, the radial displacement of the distal end of the needle with respect to the longitudinal center axis 37 will increase.

For example, referring now to FIG. 5B, for a needle assembly in which the sheath 80 includes a first and a second curvature mechanism 82 and 84, respectively, having identical interior angles 86, bending the needle at the second curvature mechanism 84 will result in a greater radial displacement of the tip 30b of the corresponding needle as the second curvature mechanism 84 is displaced a greater distance from the tip of the sheath 80 then is the first curvature mechanism 82. Although the needle assemblies of FIGS. 5A-5C are depicted as having first and second curvature mechanisms, it is contemplated that any desired number of curvature mechanisms (e.g., 3, 4, or 5 curvature mechanisms) can be provided.

Referring now to FIGS. 6A, 6B, 6C, and 7, a needle assembly 91 including a needle sheath 91 having an integrated curvature mechanism in accordance with an alternative embodiment of the present disclosure is shown. The needle assembly 90 includes an elongated hollow needle 30 including a proximal end 30a that is secured to a needle hub 32 and distal end 30b or tip that includes a bevel 34. A stylet (not shown) is received within the hollow interior of the needle 30, and a stylet hub 35 enables the practitioner to withdraw the stylet once the distal end 30b of the needle 30 has been positioned as desired. Similarly, a needle hub 32 enables the user to grip the needle hub 32 and direct the movement of the distal end 30b of the needle to its intended position. A sheath 91 is provided to maintain the sterile environment for the needle 30 and prevent unintended damage to the needle and/or injuries caused by the sharpened distal end 30b. In the embodiment shown, the sheath 91 comprises a substantially hollow body portion that is configured to slidably receive the needle 30 therein and facilitate the bending of the tip 30b of the needle 30 to a desired angle while preventing injury and contamination of the needle.

Referring specifically to FIG. 7, the distal end 90b of the sheath 90 includes a plurality of grooves 92 that form the curvature mechanism of the sheath 90. The grooves 92 depend inwardly from an outer surface of the sheath 90 in the vicinity of the distal end 30b of the needle 30. A pair of pressure pads 94a and 94b are formed on opposite sides of the plurality of grooves 92. The pressure pads 94a and 94b are slightly thicker than the remainder of the side wall of the sheath 90 and are configured for the practitioner to exert force thereon with the thumbs or forefingers. Optionally, the pressure pads 94a, 94b can comprise a different material (e.g., rubber) than adjacent portions of the sheath 90. As shown, the distal end 30b of the needle includes a bevel 34 having a heel 36 and an apex 38 that is disposed toward the side of the sheath 90 on which the plurality of grooves 92 is formed. This positioning of the bevel 34 is desired due to the fact that the bend imparted on the needle is required to be on the side of the heel 36 of the bevel 34. When the user wants to impart the bend on the distal end 30b of the needle 30, the user may position the thumbs or forefingers of their left and right hands on the pressure pads 94a and 94b on opposite sides of the grooves 92. The user next bends the two portions of the sheath 12 inwardly until the desired amount of the bend is achieved. The user may now relieve force on the sheath 91 and the metal needle 30 will remain in the bent position with the distal end 30b forming the desired angle with the proximal end 30a.

Although the foregoing invention has been described in some detail by way of illustration and example for purposes of clarity of understanding, certain changes and modifications may be practiced within the scope of the appended claims.

What is claimed is:

1. A needle assembly including:
an elongated needle including a proximal end and a distal end; and
an elongated sheath configured to slidably receive the elongated needle therein, the elongated sheath including a top surface, a bottom surface, a proximal end, a distal end, and at least one joint portion formed adjacent or proximate the distal end of the sheath,
wherein the at least one joint portion is disposed adjacent or proximate the distal end of the sheath so that a bend in the elongated sheath at the at least one joint portion produces a bend in the needle adjacent or proximate the distal end thereof.

2. The needle assembly of claim 1, wherein the at least one joint portion includes an elongated groove that is formed in the top wall of the elongated sheath, and a longitudinal axis of the elongated groove is perpendicular to a longitudinal axis of the elongated needle.

3. The needle assembly of claim 2, wherein the elongated groove depends inwardly with respect to a planar outer surface of the top wall of the sheath.

4. The needle assembly of claim 3, wherein the elongated groove is V-shaped groove having a first side wall and a second side wall that together define an internal angle of less than 20° and greater than zero degrees.

5. The needle assembly of claim 4, wherein the internal angle defined between the first side wall and the second side wall of the elongated groove is less than 10° and greater than zero degrees.

6. The needle assembly of claim 3, wherein the distal end of the elongated needle further comprises a primary bevel disposed toward the top wall of the sheath so that a heel of the primary bevel is closer to the top wall of the sheath than is an apex of the primary bevel.

7. The needle assembly of claim 6, wherein the elongated needle further comprises a pair of secondary bevels formed in a face surface of the primary bevel at the apex of the primary bevel.

8. The needle assembly of claim 6, further comprising an elongated stylet disposed within an interior of the elongated needle.

9. The needle assembly of claim 2, wherein the joint portion of the elongated sheath further comprises an elongated groove formed in the bottom wall of the sheath opposite the elongated groove formed in the top wall of the sheath.

10. The needle assembly of claim 2, wherein the at least one joint portion of the sheath is disposed between 1.5 and 0.5 cm from the distal end of the elongated sheath.

11. The needle assembly of claim 2, further comprising an injection device in fluid communication with the proximal end of the elongated needle.

12. The needle assembly of claim 2, wherein the at least one joint portion comprises a first joint portion formed a first distance from the distal end of the sheath and a second joint portion formed a second distance from the distal end of the sheath, wherein the first joint portion is disposed between the second joint portion and the distal end of the sheath.

13. The needle assembly of claim 12, wherein:
the first joint portion comprises a first V-shaped elongated groove defined by a first and a second side wall intersecting at a first internal angle,
the second joint portion comprises a second V-shaped elongated groove defined by a first and a second side wall intersecting at a second internal angle, and
the first internal angle is greater than the second internal angle.

14. The needle assembly of claim 2, wherein the proximal end of the elongated sheath is cylindrical and the distal end of the elongated sheath is rectangular so that top surface and the bottom surface are substantially parallel at the distal end of the elongated sheath.

15. A sheath assembly for use with an elongated needle, comprising:
an elongated sheath configured to slidably receive the elongated needle therein, the elongated sheath including a top surface, a bottom surface, a proximal end, a distal end, and at least one joint portion formed adjacent or proximate the distal end of the sheath,
wherein the elongated needle is slidably received in the elongated sheath, and
wherein the at least one joint portion is disposed adjacent or proximate the distal end of the sheath so that a bend in the elongated sheath at least one joint portion produces a bend in the needle adjacent or proximate the distal end thereof.

16. The sheath assembly of claim 15, wherein the at least one joint portion includes an elongated groove that is formed in the top wall of the elongated sheath, and a longitudinal axis of the elongated grove is perpendicular to longitudinal axis of the elongated needle.

17. The sheath assembly of claim 16, wherein the elongated groove depends inwardly with respect to a planar outer surface of the top wall of the sheath.

18. The sheath assembly of claim 17, wherein the elongated groove is V-shaped having a first side wall and a second side wall that define an internal angle of less than 20°.

19. The sheath assembly of claim 18, wherein the internal angle defined between the first side wall and the second side wall of the elongated grove is less than 10°.

20. The sheath assembly of claim 17, wherein the distal end of the elongated needle further comprises a bevel disposed toward the top wall of the elongated sheath so that a heel of the bevel is closed to the top wall of the sheath than is an apex of the bevel.
